(19) United States
(12) Patent Application Publication
(10) Pub. No.: US 2026/0097082 A1
(43) Pub. Date: Apr. 9, 2026

(54) HUMAN HAIR FOLLICLE-DERIVED CONDITIONED MEDIA AND METHOD OF MAKING SAME

(71) Applicant: ACORN BIOLABS, INC., Toronto (CA)

(72) Inventors: Drew Wesley Taylor, Toronto (CA); Muneera Walid Fayyad, North York (CA); Amatullah Khuzaima Fatehi, North York (CA)

(21) Appl. No.: 19/113,090

(22) PCT Filed: Sep. 27, 2023
(86) PCT No.: PCT/CA2023/051281

§ 371 Date: Mar. 19, 2025

Related U.S. Application Data
(60) Provisional application No. 63/410,903, filed on Sep. 28, 2022.

(51) Int. Cl. A61K 35/36; A61K 8/98; A61K 35/19; A61P 17/02; A61Q 7/00; C12N 5/00; C12N 5/071

(52) U.S. Cl. CPC A61K 35/36; A61K 8/985; A61K 35/19; A61P 17/02; A61Q 7/00; C12N 5/0056; C12N 5/0627; C12N 5/56

(57) ABSTRACT

A method of preparing hair follicle-derived conditioned medium which comprises one or more bioactive factors, wherein the hair follicle is retrieved from a subject non-invasively, cultured in a culture medium for a duration sufficient for a hair follicle-derived cell to secrete the one or more bioactive factors to condition the medium. A hair follicle-derived conditioned medium comprising one or more bioactive factors secreted by a hair follicle-derived cell retrieved from a subject non-invasively. A composition comprising said conditioned medium. Use of said conditioned medium and composition for therapeutic or cosmetic treatment.

### FIELD OF THE INVENTION

The present specification relates generally to secretome-based therapies, and more particularly to conditioned media prepared non-invasively using hair follicle.

### BACKGROUND OF THE INVENTION

Secretome-based therapies have gained much interest in therapeutic and cosmetic fields for the treatment of various conditions, including hair loss, skin conditions and sport-related injuries. These effects are mediated by the action of an array of secreted bioactive factors, such as cytokines and growth factors, collectively referred to as the “secretome”. Substantial evidence from previous studies acknowledges that harnessing the secretome of stem cells is of great therapeutic promise, albeit the need for highly invasive tissue collection methods and poor scalability of the product are major limitations that have been neglected in the prior art.

Different tissues where stem cells reside have been investigated for secretome therapies in the prior art. These include bone marrow, adipose tissue, amniotic fluid or umbilical cord, all of which are collected via highly invasive methods. Prior art examples of conditioned media therapeutics that exist currently in the market include BabyStem™ from the bone marrow, BioCM™ from the amniotic fluid, and several compositions from adipose-tissue such as AAPE™, Exomide™, TheraStem™, Stem C'rum™, among others. In addition to tissue invasiveness, all said products are allogeneic therapies harvested from continually expanded and cultured cells with little or no disclosed information about the cellular and donor origin of these cells. It is well established that cells could lose or acquire various intrinsic properties with prolonged passaging, including an altered proteomic profile. We now know that contents of secretome vary tremendously among donors, which imposes the biggest challenge against allogeneic therapies. Although secretome therapies generally mitigate the safety risks inherent in administering large amounts of viable stem cells, allogeneic sources remain a barrier to the safety of these therapies.

Another factor that is neglected in prior art secretome therapies is the inability to rule out the presence of animal (xenogenic) serum residue in the composition of the final product. Animal-derived components, such as fetal bovine serum, are commonly used to cultivate cells and tissues in culture. This raises reliability and biosafety concerns. Although most prior art secretome therapies switch to xenogenic-free medium shortly before conditioned media collection, use of animal products at any stage during the cell cultivation process remains problematic. The main concern with using animal components is the introduction of contaminants into the process and thus potentially into the final therapeutic product, which deems these inappropriate for clinical or therapeutic use. Using xenogenic-free reagents throughout the process of secretome production is therefore necessary to warrant clinical safety for human subjects.

The use of a completely serum-free media confers many advantages, including consistency in formulations and biosafety. However, it can lead to suboptimal growth and cellular metabolism, and there is an unmet need for alternative methods to enrich secretome compositions in a manner that is equally safe for human use.

Accordingly, there is a need for novel secretome therapies and preparation methods to overcome the limitations of existing secretome therapies. The present invention discloses conditioned media that may be used for autologous secretome therapies, prepared non-invasively using hair follicles and utilizing a xenogenic-free system to expand cells.

### BRIEF SUMMARY OF INVENTION

The present invention relates to methods of producing conditioned medium comprising one or more bioactive factors, non-invasively using hair follicles. While stem cells located in the dermal papilla of hair follicle have been extensively studied for their role in immunomodulation and wound healing, these cells have been historically retrieved using invasive means including through follicular unit extraction (FUE), a technique used in hair transplantation. In various aspects, the present invention is based on the discovery that cells located in upper regions of hair follicle, which can be consistently retrieved non-invasively, are capable for use and a great source for producing secretome therapies. Additionally, not only are the cells located in the upper regions of the follicle able to be consistently collected non-invasively, but they are also more readily cultured directly from the follicle and without the need of digestive enzymes. Compared to other stem cell tissue resources, the human hair follicle, in the anagen state, is an abundant tissue resource that is easily and non-invasively accessible throughout adulthood. Within the outer root sheath of the hair follicle resides a heterogenous variety of cell types including an endogenous population of stem cells, responsible for hair growth and development. Hence, there is value in utilizing this tissue resource for the production of secretome therapies.

According to one embodiment, the preparation methods of the present invention comprise obtaining hair follicle through non-invasive means, culturing the hair follicle in growth culture medium, such as but not limited to autologous platelet lysates, for a suitable period of time for hair follicle-derived cell to secrete one or more molecules into the growth culture medium, thus conditioning the medium, and collecting the conditioned medium for further processing or immediate use.

In certain embodiments, the preparation methods of the present invention non-invasively produce xenogenic-free conditioned medium that may be used for autologous secretome therapy, thereby providing a GMP-ready, scalable, quality-controlled protocol for the generation of a clinic-ready therapeutic product.

The present invention also relates to conditioned media comprising one or more bioactive factors prepared using the method of the present invention.

The present invention also relates to conditioned media comprising one or more bioactive factors secreted by a hair follicle-derived cell obtained from a subject.

The present invention further relates to compositions comprising the conditioned media of the present invention and other diluents, excipients, or carriers.

The present invention also relates to use of the conditioned media and compositions of the present invention for autologous or allogeneic secretome therapies.

Other and further aspects and features of the present disclosure will become apparent during the course of the following discussion and by reference to the accompanying drawings.

### BRIEF DESCRIPTION OF DRAWINGS

For a better understanding of the various implementations described herein and to show more clearly how they may be carried into effect, reference will now be made, by way of example only, to the accompanying drawings in which:

FIG. 1 depicts one preferred example of a process flow diagram for the preparation of conditioned medium using hair follicle and use of such conditioned medium for, as example, the treatment of hair loss.

FIG. 2 depicts phase-contrast images of a hair follicle and its derived cells expanding out of the follicle, in growth culture medium, at 5 and 12 days.

FIG. 3 depicts expression of keratinocyte markers in a subpopulation of hair follicle-derived cells.

FIG. 4 depicts expression of mesenchymal stem cell markers in a subpopulation of hair follicle-derived cells.

FIG. 5 depicts human antibody cytokine array analysis showing levels of analytes in hair follicle-derived conditioned medium, as an embodiment of the present invention, in comparison to platelet-rich plasma prepared from the same subject.

FIG. 6 depicts an in vitro wound-healing (scratch test) assay of human skin cells cultured in standard growth medium compared to standard growth medium with hair follicle-derived conditioned medium, an embodiment of the present invention.

### DETAILED DESCRIPTION

The description that follows, and the embodiments described therein, is provided by way of illustration of an example, or examples, of particular embodiments of various aspects of the present disclosure. These examples are provided for the purpose of explanation, and no limitation of scope of the invention is thereby intended. Well-known methods, procedures and components have not been described in detail so as not to obscure the exemplary aspects described herein.

### Definitions

Unless defined otherwise, all terms and phrases used herein include the meanings that the terms and phrases have attained in the art, unless the contrary is clearly indicated or clearly apparent from the context in which the term of phrase is used.

As used herein, the term “hair follicle” refers to an epidermal structure that begins at the surface of the epidermis and anchors the hair shaft to the skin. It contains cells, connective tissue and surrounds the root of the hair. The cyclic growth of the hair is driven by a population of stem cells that reside within the hair follicle. Hair follicles could be obtained using a variety of non-invasive methods, such as plucking, follicular unit extractions or others.

As used herein, the term “anagen” refers to the active phase of the hair growth cycle, marked by rapid division and differentiation of cells, including stem cells residing in the bulge region of the outer root sheath.

As used herein, “stem cell” refers to a cell that has the capacity for self-renewal, i.e., the ability to sustain numerous cycles of cell division while still maintaining a non-terminally differentiated state.

As used herein, the term “mesenchymal stem cell” is a multipotent cell that can differentiate into a variety of cellular lineages, including osteocyte, adipocyte and chondrocyte. It is found to have a characteristic set of markers, with additional functions including secretion of immunomodulatory factors. According to certain preferred embodiments, the conditioned medium and composition of the present invention contain one or more bioactive factors secreted by mesenchymal stem cell from the cultured hair follicle.

As used herein, the term “keratinocyte” refers to a highly specialized cell within the epidermal layer of the skin. It has a structural role, a protective role, and an immunomodulatory role within the skin. It is responsible for restoring the epidermis following injuries by migrating to the wound and secreting specific factors to initiate interactions between different cell types for a successful healing process. According to certain preferred embodiments, the conditioned medium and composition of the present invention contain one or more bioactive factors secreted by keratinocyte from the cultured hair follicle.

As used herein, persons skilled in the art may generally understand the term “secretome” to mean the totality of all bioactive substances released by cells. These may include growth factors, cytokines, and other proteins, nucleic acids, lipids, or hydrocarbons. As part of the secretome, some bioactive molecules are secreted within membrane-bound vesicles, such as exosomes (see, for example, reference [1]). The secretome from stem cells can exert a broad array of important physiological functions by acting as molecular messengers that communicate between different cell types. Various molecules within the secretome can signal wound-healing, cell proliferation, recruitment of extracellular matrix components, immunomodulation, and the like (see, for example, reference [1]). Therefore, secretome is believed to hold great potential for tissue repair and regeneration.

The term “autologous” as used herein, refers to cells, tissues, fluids, and the like obtained from the same subjection; for example, an autologous therapeutic product is a product in which donor cells or tissues are obtained from the same subject that is also the recipient of the product, and thus the donor cells or tissues are genetically identical to the recipient.

The term “allogeneic” as used herein, refers to cells, tissues, fluids, and the like that are genetically dissimilar; for example, an allogeneic therapeutic product is a product in which cells or tissues are donated by a donor subject and are therefore genetically distinct from the recipient of the product.

As used herein, the term “substantially free” means that the presence of a particular component is either not detected using known assays, or if it is detected, it is only present in an amount that is at least 99% free of the component.

As used herein, persons skilled in the relevant art may generally understand the term “conditioned media” to be culture medium (or extract, part, or fraction thereof) in which one or more cells of interest (such as hair follicle-derived cells) have been cultured. The culturing of said cells may result in secretion of one or more molecules and/or bioactive factors into the extracellular space (such as into a culture medium) over a period of time, thus conditioning the culture medium. A conditioned media may be left unpurified or further processed. For example, components of a conditioned medium may further comprise one or more substances that are not secreted from a cell (such as additives and nutrients contained in the initial growth culture medium). Alternatively, a conditioned medium does not comprise one or more substances or trace amounts thereof that are not secreted from a cell.

As used herein, the term “cell culture” refers to cells grown under controlled conditions outside the natural environment of these cells.

The terms “culture medium”, “growth medium”, and “growth culture medium”, as used herein, refer to a composition that is intended to support the growth and survival of cells or tissues outside their natural environment and is often in liquid form.

As used herein, the term “basal medium” refers to an unsupplemented synthetic culture medium that may contain buffers, one or more carbon sources and salts. Depending on the cell type and culturing conditions, basal media may be supplemented with additives and growth factors, including but not limited to, additional buffering agents, amino acids, antibiotics, proteins, growth factors and other nutrients essential for promoting growth or survival of particular cells of interest.

As used herein, the term “platelet-rich plasma” or “PRP” refers to a concentration of a subject's own plasma, rich in platelets, and derived from whole blood, centrifuged to remove red blood cells. It has been used by those skilled in the art as an emerging treatment, often in the form of topical applications or injections, to accelerate the healing of different tissues within the body, such as but not limited to, tendons and ligament injuries, hair and skin rejuvenation, arthritis-related pain, retinal conditions and other applications. PRP therapies vary tremendously in their preparation methods depending on what is optimal for each specific clinical indication. For example, some procedures may further comprise an activation step, whereby plasma and platelets are subjected to an additive, such as but not limited to thrombin or calcium chloride, to stimulate granulation of platelets and release of growth factors into the medium, referred to thereafter as “platelet lysate”. The term “derivative of PRP” is a collective term that encompasses platelet lysates, or any other form of PRP that has been further processed after centrifugation or subjected to additives to enhance efficacy for cell culture.

As used herein, the term “effective amount” refers an amount of a compound, molecule, component, composition, conditioned media or a dilution thereof that is sufficient to achieve the desired effect (e.g., a therapeutic effect, limiting an insult or injury to one or more tissues as experienced by the subject).

As used herein, the term “lyophilization” refers to the process in which water is omitted from a product after it is frozen and placed in a vacuum-like equipment, allowing the water to change directly from a solid phase to vapor without passing through a liquid phase. It is a method commonly used to preserve perishable biological materials, to extend shelf life or stabilize the material for transport purposes.

### Method of Preparation

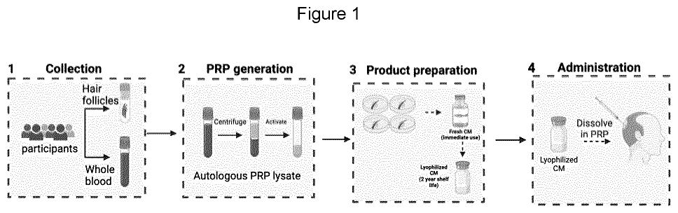

Attention is directed to FIG. 1, which depicts an exemplary process flow that may be used with various non-limiting embodiments of the present invention, from sample collection, to conditioned medium preparation, to use of such conditioned medium.

According to certain embodiments, the present invention provides a method of preparing conditioned medium comprising one or more bioactive factors, the method comprising: obtaining hair follicle; culturing the hair follicle in a culture medium for a duration sufficient for a hair follicle-derived cell to secrete the one or more bioactive factors, thereby conditioning the culture medium; and collecting the conditioned medium.

According to an embodiment of the present invention, hair follicle is obtained from a subject using non-invasive methods such as plucking and follicular unit extraction. In a more particular embodiment, the extracted hair follicle may be cultured immediately. In another embodiment, the extracted hair follicle may be transported in the transport composition of WO2020/024045A1 to the intended facility. In yet another embodiment, the extracted hair follicle may be cryopreserved in the cryopreservation composition of WO2020/024045A1 for variable amounts of time before thawing and cultivation.

It is known that PRP-derived supplements have been used directly as a safe and efficacious treatment for many clinical indications, in addition to its utilization for culturing cells or tissues in vitro as an alternative to animal serum or serum-free supplements (see, for example, reference [2]). Thus, in a more particular embodiment, the culture medium used in the preparation of the conditioned medium comprises PRP or derivative of PRP (e.g., platelet lysate). In a preferred embodiment, the culture medium comprises about 1% to about 30% w/w platelet lysate, preferably about 5% to about 25% w/w platelet lysate, and more preferably, about 20% w/w platelet lysate.

The development of autologous conditioned media therapies could warrant greater clinical safety and regulatory and commercial desirability of the clinical product. Thus, in a particular embodiment, the PRP or derivative of PRP (e.g., platelet lysate) is autologous to the hair follicle used in conditioning the culture medium. According to certain embodiments, whole blood is withdrawn from a subject and PRP is prepared by centrifugation for a suitable period of time in conditions standard in the art. In one embodiment, PRP may be activated by the addition of chemicals, such as but not limited to thrombin or calcium chloride to enhance release of growth factors into said composition, forming platelet lysate. In a preferred embodiment, the concentration of said PRP activation chemicals (e.g., thrombin and/or calcium chloride) is about 20 mM to about 30 mM. Alternatively, according to certain embodiments, the culture medium may comprise supplements such as blood substituents, human recombinant growth media, or other commercially available xenogenic-free nutrients.

According to certain embodiments, no animal serum or any other animal components are used in the preparation of the conditioned medium. In one embodiment, a number of human hair follicles are cultured in a predominantly autologous, completely xenogeneic free culture medium.

According to certain embodiments, the culture medium for culturing hair follicle may comprise basic media known in the art to which the present invention pertains without limitation. The basal medium may be prepared by artificial synthesis, or a commercially prepared medium may be used. Examples of commercially available medium include, but are not limited to, Dulbecco's Modified Eagle's Medium (DMEM), Minimal Essential Medium (MEM), Basal Medium Eagle (BME), RPMI 1640, F-10, F-12, alpha-Minimal Essential Medium (alpha-MEM), Glasgow's Minimal Essential Medium (G-MEM), and Modified Dulbecco's Medium (Isocel's Modified Dulbecco's Medium). In some embodiments, the culture medium comprises Knock Out Dulbecco's Modified Eagle Medium/F-12, MEM, BME, platelet lysate, or other xenogenic-free medium known in the art.

According to certain embodiments, the culture media may comprise additional growth factors, such as but not limited to, basic fibroblast growth factor (b-FGF) at effective amounts to stimulate growth and expansion of cells. In further embodiments, the culture media may also comprise additional antibiotics to prevent bacterial contamination, such as penicillin-streptomycin, at amounts that are standard in the art.

In an embodiment, the culture media comprises components selected from Knock Out Dulbecco's Modified Eagle Medium/F-12, MEM, BME, or other xenogenic-free medium known in the art, autologous platelet lysate at a concentration of about 5% to about 25% w/w, penicillin and/or streptomycin, or any combination thereof.

According to certain embodiments, the hair follicle is cultured under optimal conditions to stimulate cellular expansion. In some embodiments, the hair follicle is cultured at a temperature between about 20° C. and about 50° C. In some preferred embodiments, the hair follicle is cultured at a temperature between about 30° C. and about 40° C., and more preferably at about 37° C.

According to certain embodiments, the hair follicle is maintained at CO₂ percentage between about 2% and about 10%, preferably at about 5% CO₂.

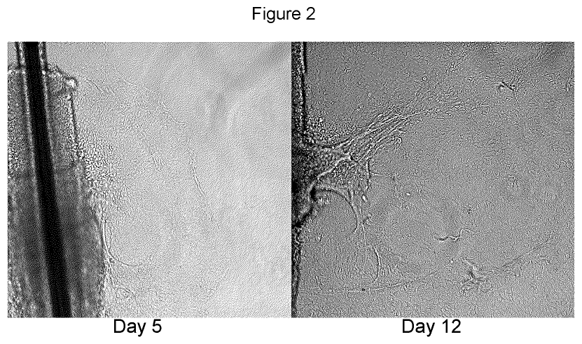

According to certain embodiments, the hair follicle may be plated in culture in the growth culture media for a time period of about one to about six weeks, preferably about four weeks. Attention is directed to FIG. 2, which depicts a phase-contrast microscope image of hair-follicle-derived cells outgrowing from the outer root sheath of a hair follicle in culture, at day 5 and day 12.

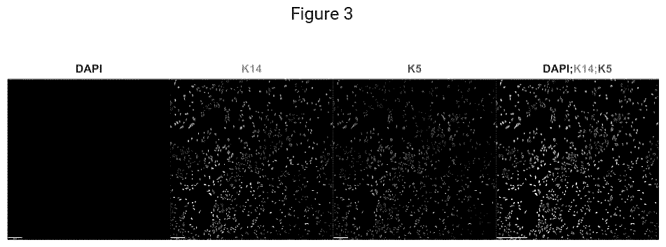

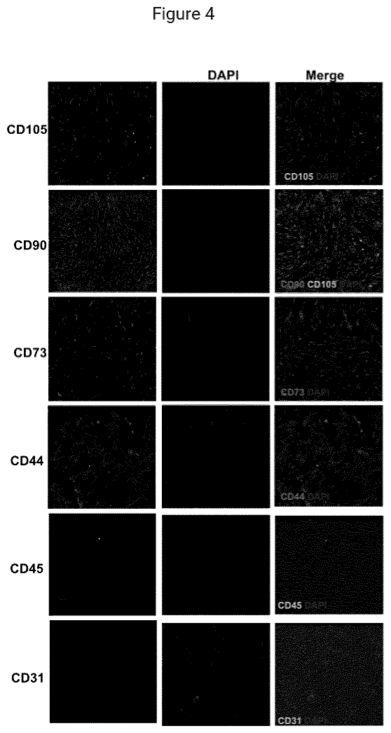

According to certain embodiments, during cultivation stage of the hair follicle in the growth culture medium, a heterogeneous population of cells is present, which may include cells such as but not limited to keratinocyte, mesenchymal stem cell, fibroblast, hair-associated pluripotent cell, dermal papilla cell, among other primary somatic or stem cell types, cultivated according to standard procedures as known to those skilled in the art. For example, attention is directed to FIG. 3, which depicts immunofluorescent staining showing prominent expression of basal keratinocyte markers KRT14 and KRT5 in expanded hair follicle-derived keratinocytes. Another example cell type is illustrated in FIG. 4, which depicts immunofluorescence staining of mesenchymal stem cells isolated from the hair follicle for standard mesenchymal-associated markers known in the art; the cells express a typical immunophenotypic profile of mesenchymal stem cells (CD90+CD105+CD73+CD44+ and CD45−CD31−). The cultivation of different cells and expression of paracrine factors may influence the therapeutic efficacy of the conditioned medium or composition containing such conditioned medium described herein.

According to certain embodiments, the hair follicle is cultured in the culture medium to about 80% to about 90% confluency in order to condition the culture medium. In one embodiment, the conditioned medium is collected at regular intervals between about 12 hours and about 96 hours from the start of the culture.

According to certain embodiments, the conditioned medium undergoes further processing. In one embodiment, the conditioned medium is centrifuged and filtered to purify the conditioned medium and remove cellular debris. In a more particular embodiment, the conditioned medium is filtered using a 0.22 μm filter to ensure removal of cellular material. In another embodiment, the conditioned medium is cryopreserved, frozen, or lyophilized. In a more particular embodiment, an effective amount of lyophilizing agent, such as sucrose, is added to the conditioned medium to act as a protein stabilizer and protect the conditioned medium during the process of freeze drying; determination of the optimal lyophilizing agent and the effective amount of such agent is within the routine knowledge and skill of persons skilled in the art. In a further embodiment, the lyophilized conditioned medium is suspended in a suspension mixture comprising one or more of: saline, hyaluronic acid, silicone gel, petroleum jelly, PRP, derivative of PRP, platelet lysate, and a pharmaceutically acceptable excipient.

According to a preferred embodiment, the method of preparing conditioned medium comprising one or more bioactive factors includes the following steps:
extracting hair follicle from a subject non-invasively through plucking or through follicular unit extraction;
culturing the hair follicle in a xenogenic-free culture medium or autologous PRP or a combination of the two to induce cellular expansion;
collecting the conditioned medium upon confluency of the cultured cells; and
optionally further processing the conditioned medium with centrifugation, filtration, lyophilization or other steps to ensure elimination of cellular material, purity and stability of the final product.

### Conditioned Medium

According to certain embodiments, the present invention also provides a conditioned medium comprising one or more bioactive factors secreted by a hair follicle-derived cell obtained from a subject. In some embodiments, such conditioned medium is prepared using the preparation methods described herein.

According to certain embodiments, the one or more secreted factors may include regenerative factors which could include proteins (e.g., growth factors, cytokines, chemokines), nucleic acids (e.g., miRNA), polysaccharides (e.g., hyaluronic acid), and/or a combination thereof, either bound within or separate from extracellular vesicles (e.g., exosomes).

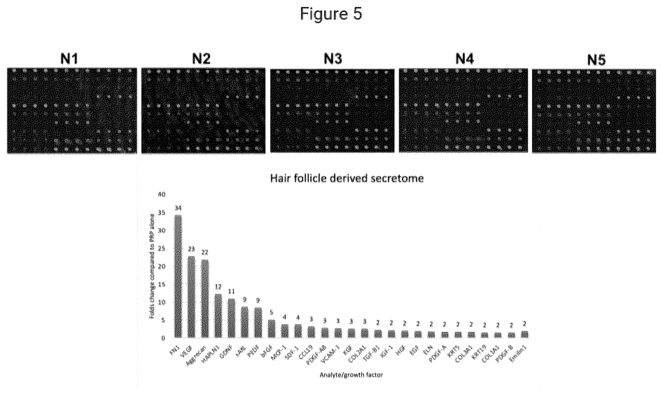

According to certain embodiments, the one or more bioactive factors may include but not limited to, hyaluronic acid, elastin, HAPLN1, collagens (e.g., COL1A1, COL2A1, COL3A1), keratins (e.g., KRT5, KRT19), fibronectin, EMILIN1, KGF, PDGF, bFGF, TGF-31, HGF, EGF, VEGF, CCL19, sAXL, SDF-1, VCAM-1, MCP-1, IGF-1, GDNF, BDNF, NRG2, PDGF, Interleukins (e.g., IL-1, IL-2, IL-6) or any combination thereof, at concentrations varying among subjects and ranging from an amount of 0 μg/mL to 1000 μg/mL. Attention is directed to FIG. 5, which depicts an antibody-based cytokine array analysis showing the levels of a number of bioactive factors detected in a non-limiting embodiment of the conditioned media.

According to certain embodiments, the present invention further provides a composition comprising the conditioned medium disclosed herein and one or more of: saline, hyaluronic acid, PRP, derivative of PRP, platelet lysate, a pharmaceutically acceptable excipient, carrier, and diluent. In a further embodiment, the composition is formulated into dosage forms selected from injection, oily suspension, hydrogel, nanogel, ointment, cream, serum, emulsion, spray, patch, gel, drop, or any combination thereof. In a more particular embodiment, the invention provides a pharmaceutical or cosmetic composition comprising conditioned medium, extracellular vesicles, autologous PRP, hyaluronic acid, or a combination thereof.

According to certain embodiments, the conditioned medium or composition is substantially free of animal serum or any animal components. Animal serum and other animal-derived components are widely used in cell and/or tissue culture as a source of growth factors, hormones, amino acids, lipids and other nutrients. These are associated with biosafety concerns and subject to batch-to-batch variability. Although most prior art secretome therapies switch to serum-free culture media before collection, use of animal products at any stage during the cell cultivation process remains a concern. Introduction of contaminants into the process and thus potentially into the final therapeutic product is a major safety concern upon translation into clinical application.

According to certain embodiments, the conditioned medium or composition comprises additional soluble components, including but not limited to growth factors from the growth culture medium intended to stimulate the growth and expansion of hair follicle-derived cells cultured within. In one embodiment, such growth factors originate from the PRP or derivative of PRP contained in the culture medium. In another embodiment, the growth factors are added to the culture medium, including but not limited to about 0.01 to about 10 ng/ml recombinant human basic fibroblast growth factor, or other recombinant, synthetic, or human-derived growth factors.

According to certain embodiments, the conditioned medium or composition is substantially free of whole cells or cellular debris. In one embodiment, the conditioned medium is a cell-free secretome product prepared from a heterogeneous population of primary cells cultured from the human hair follicle.

According to certain embodiments, the conditioned medium or composition is autologous.

According to a preferred embodiment, the conditioned medium or composition is autologous, xenogeneic-free, prepared by culturing human hair follicle in xenogenic-free conditions to stimulate the growth of hair follicle-derived cells. In another preferred embodiment, the conditioned medium or composition is prepared by culturing human hair follicle in PPR or derivative of PRP to stimulate the growth of hair follicle-derived cells.

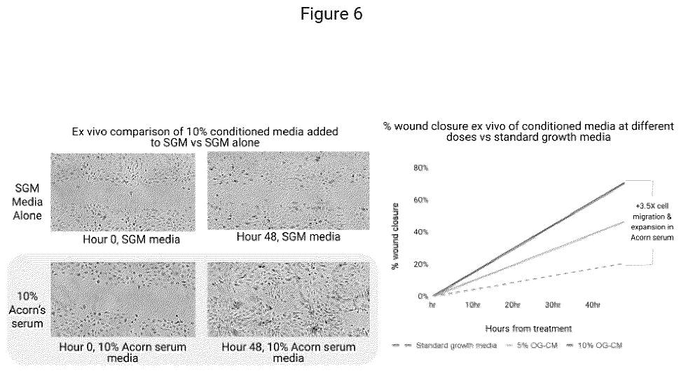

According to certain embodiments, the conditioned medium or composition may be used for therapeutic or cosmetic purposes, including for wound healing, stimulating soft or connective tissue repair/regeneration (e.g., hair regeneration, skin rejuvenation, reduction or prevention of signs of skin aging, or treatment of joint-related conditions) in, or delivery of the one or more bioactive factors to, a targeted tissue site. Attention is directed to FIG. 6, which compares wound healing via a scratch assay in human skin cells cultured in standard growth medium versus human skin cells cultured in standard growth media with a preferred embodiment of the conditioned medium added.

In an embodiment, a therapeutic effective amount of conditioned medium or composition is administered to a subject, or cells or tissues of such subject, for a suitable period of time. In a preferred embodiment, the conditioned medium or composition is used for autologous therapy in the subject from which hair follicle was collected. In another embodiment, the conditioned medium or composition may be used for allogeneic therapy in a subject, wherein the conditioned medium is prepared from hair follicle of a healthy donor.

According to certain embodiments, the conditioned medium may be used directly and freshly after preparation or lyophilized for storage. In one embodiment, the lyophilized conditioned medium may be stored at room temperature for a period of about 12 weeks or at about 4° C. for up to about 24 months. In another embodiment, the lyophilized conditioned medium may be mixed with a solid or a liquid carrier including PRP, hyaluronic acid, saline, or a cream or serum for topical or injected application.

### Kit

According to certain embodiments, the present invention provides a kit comprising a culture medium suitable for culturing a hair follicle and user instructions for a subject to prepare hair follicle-derived conditioned medium.

### EXAMPLES

The invention will be further illustrated by the following non-limiting examples. These examples are set forth to aid in the understanding of the disclosure but are not intended to, and should not be construed to, limit the scope of the invention in any way.

The examples do not include detailed descriptions of conventional methods that would be well established and known to persons skilled in the art.

### Example 1—Preparation of Conditioned Medium by Cultivation of Hair Follicle in Autologous Platelet Lysate

This example sets out a preferred method of preparing conditioned medium comprising one or more bioactive factors using hair follicle in accordance with an aspect of the present invention.

Hair follicles are rinsed thoroughly with antibiotic-antimycotic solution, cut to 1-2 mm distal to the outer root sheath and then plated on culture vessels pre-coated with CELLstart substrate. The hair follicles are then incubated with 5% CO₂, 5% O₂, at 37° C. for a time period between two weeks and four weeks.

Culture growth medium consisting of 20% autologous platelet lysate diluted in basal media is added to plated hair follicles. Culture growth medium is changed every two days to maintain viability of the cells.

Once hair follicle-derived cells reach 70-90% confluency, the conditioned medium is collected, centrifuged at 300×g for 5 minutes, and the supernatant is filter-sterilized through a disposable sterile 0.22 um filter system.

The resulting solution is then aliquoted into sterile vials and either stored at 4° C. for immediate use, or lyophilized to facilitate long-term storage.

### Example 2—Ex Vivo Wound Healing Assay Comparing Hair Follicle-Derived Conditioned Medium and Standard Growth Medium

This example sets out an ex vivo wound healing assay to show the effect on wound healing of hair follicle-derived conditioned medium compared to standard growth medium alone as control.

Subject skin cells were cultured in 12 well-plates until confluency. Once confluent, a scalpel was used to create a defect by scraping off cells from the surface of the plate. This resulted in an artificial wound where cells were absent from being scraped off.

The artificially wounded skin cells were then cultured in either a standard growth media, known to promote proliferation and migration, or 10% conditioned medium in the standard growth media prepared in accordance with the method described in Example 1.

The artificially wounded skin cells were observed and measured for wound closure, cell migration and expansion. As shown in FIG. 6, the skin cells cultured in 10% conditioned medium after the introduction of artificial wound showed an increase in wound closure, through enhanced and combined cell proliferation and migration, by about 3.5 times after about only 48 hours of cultivation, compared to the control skin cells cultured in standard growth medium only. The results of this wound healing assay show improved wound healing associated with hair follicle-derived conditioned medium.

### Example 3—Hair Follicle-Derived Conditioned Medium as a Potential Treatment for Androgenic Alopecia

This example sets out a clinical trial to evaluate the efficacy of hair follicle-derived conditioned medium for the treatment of a hair loss condition such as androgenic alopecia.

Subjects who are diagnosed with androgenic alopecia are selected for this study. The following inclusion criteria are used:
Male subjects with a clinical diagnosis of androgenetic alopecia.
Subjects must agree to the following sample collection:
   at least 20 hair follicles plucked from the back of scalp, and
   a total of 30 ml of blood.

Hair follicles and blood samples are collected from subjects to allow for the preparation of autologous hair follicle-derived conditioned media, in accordance with the preparation method described in Example 1.

The resulting conditioned media are lyophilized. Lyophilized conditioned media are suspended in PRP, hyaluronic acid, saline, or any suitable solvent or carrier.

Subjects are administered an effective dose of conditioned media topically or intradermally into the site of treatment once every month for five months.

The following baseline examinations are performed throughout the study: Trichogram analysis to assess hair density and anagen induction, clinical photographs and ultrasonography to evaluate dermal thickness and echogenicity.

### Example 4—Hair Follicle-Derived Conditioned Medium as a Potential Treatment for Improved Wound Healing and Inhibition of Scar Tissue

This example sets out a clinical trial to evaluate the efficacy of hair follicle-derived conditioned medium for improved and accelerated wound healing post elective plastic surgery.

Subjects who are scheduled for an abdominoplasty (tummy tuck) to remove excess skin and fat from the abdomen:
Female subjects with a scheduled surgical date for abdominoplasty.
Subjects must agree to the following sample collection:
   at least 20 hair follicles plucked from the back of scalp one month prior to surgery.

Hair follicles are collected from subjects to allow for the preparation of autologous hair follicle-derived conditioned media, in accordance with the preparation method described in Example 1.

The resulting conditioned media are lyophilized. Lyophilized conditioned media are suspended in hyaluronic acid.

Subjects are administered an effective dose of conditioned media, once daily over 7 days, topically onto one half of the abdominoplasty. Subjects are administered hyaluronic acid, once daily over 7 days, topically onto the second half of the abdominoplasty. This provides a split-face study with the treated and control sites on each subject.

The following baseline examinations are performed throughout the study: clinical photographs and ultrasonography to evaluate dermal thickness, topography, scar tissue formation, and healing time.

Persons skilled in the art will appreciate that there are yet more alternative implementations and modifications possible, and that the above examples are only illustrations of one or more implementations. The scope, therefore, is only to be limited by the claims appended hereto.

### Interpretation

It will be understood that for the purposes of this application, “at least one of X, Y, and Z” or “one or more of X, Y, and Z” language can be construed as X only, Y only, Z only, or any combination of two or more items X, Y, and Z (e.g., XYZ, XYY, YZ, ZZ).

In the present application, components may be described as being “configured to” or “enabled to” perform one or more functions. Generally, it is understood that a component that is configured to or enabled to perform a function is configured to or enabled to perform the function, or is suitable for performing the function, or is adapted to perform the function, or is operable to perform the function, or is otherwise capable of performing the function.

References in the application to “one embodiment”, “an embodiment”, “an implementation”, “a variant”, etc., indicate that the embodiment, implementation or variant described may include a particular aspect, feature, structure, or characteristic, but not every embodiment, implementation or variant necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of a person skilled in the art to affect or connect such module, aspect, feature, structure, or characteristic with other embodiments, whether or not explicitly described. In other words, any module, element or feature may be combined with any other element or feature in different embodiments, unless there is an obvious or inherent incompatibility, or it is specifically excluded.

It is further noted that the claims may be drafted to exclude any optional element. As such, this statement is intended to serve as antecedent basis for the use of exclusive terminology, such as “solely”, “only”, and the like, in connection with the recitation of claim elements or use of a “negative” limitation. The terms “preferably”, “preferred”, “prefer”, “optionally”, “may”, and similar terms are used to indicate that an item, condition or step being referred to is an optional (not required) feature of the invention.

It will be further understood that the terms “comprises,” “comprising,” “includes,” and/or “including,” when used herein, specify the presence of stated features, steps, operations, elements, and/or components, but do not preclude the presence or addition of one or more other features, steps, operations, elements, components, and/or groups thereof.

The singular forms “a”, “an”, and “the” include the plural reference unless the context clearly dictates otherwise. The term “and/or” means any one of the items, any combination of the items, or all of the items with which this term is associated. The phrase “one or more” is readily understood by persons skilled in the art, particularly when read in context of its usage.

As will be understood by persons skilled in the art, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range. Any listed range can be easily recognized as sufficiently describing and enabling the same range being broken down into at least equal halves, thirds, quarters, fifths, or tenths. As non-limiting example, each range discussed herein can be readily broken down into a lower third, middle third and upper third, etc.

### References

1. Wangler, S., Kamali, A., Wapp, C. et al. Uncovering the secretome of mesenchymal stromal cells exposed to healthy, traumatic, and degenerative intervertebral discs: a proteomic analysis. Stem Cell Res Ther 12, 11 (2021).
2. Burnouf, T., Strunk, D., Koh, M.B., & Schallmoser, K. (2016). Human platelet lysate: replacing fetal bovine serum as a gold standard for human cell propagation?. Biomaterials, 76, 371-387.
3. Walter MN, Wright KT, Fuller HR, MacNeil S, Johnson WE. Mesenchymal stem cell-conditioned medium accelerates skin wound healing: an in vitro study of fibroblast and keratinocyte scratch assays. Exp Cell Res. 2010 Apr. 15; 316(7): 1271-81.
4. Topouzi, Helena, et al. Harnessing the secretome of hair follicle fibroblasts to accelerate ex vivo healing of human skin wounds. Journal of Investigative Dermatology 140.5 (2020): 1075-1084.

1. A method of preparing conditioned medium comprising one or more bioactive factors, the method comprising:
obtaining hair follicle, the hair follicle retrieved from a subject non-invasively;
culturing the hair follicle in a culture medium for a duration sufficient for a hair follicle-derived cell to secrete the one or more bioactive factors, thereby conditioning the culture medium; and
collecting the conditioned medium.

2. The method of claim 1, wherein the culture medium comprises platelet-rich plasma (PRP), derivative of PRP, platelet lysate, or about 1 % to about 30% w/w platelet lysate.

3. The method of claim 2, wherein the PRP, derivative of PRP, or platelet lysate is autologous to the hair follicle.

4. The method of claim 2, wherein the platelet lysate is prepared by activating PRP with thrombin, calcium chloride, or any combination thereof, at a concentration of about 20 mM to about 30 mM.

5. The method of claim 1, wherein the culture medium comprises KnockOut Dulbecco's Modified Eagle Medium/F-12, Minimal Essential Medium, Basal Medium Eagle, xenogeneic-free medium, penicillin, streptomycin, about 0.01 ng/mL to about 10 ng/mL growth factor or basic fibroblast growth factor, or any combination thereof.

6. The method of claim 1, wherein the hair follicle-derived cell is a keratinocyte, mesenchymal stem cell, fibroblast, hair-associated pluripotent cell, dermal papilla cell, hair follicle-derived stem cell, hair follicle-derived somatic cell, or any combination thereof.

7. The method of claim 1, wherein the hair follicle is cultured to about 80% to about 90% confluency.

8. The method of claim 1, further comprising at least one of the following steps:
centrifuging and filtering the conditioned medium to remove cellular debris;
cryopreserving, freezing, or lyophilizing the conditioned medium;
and suspending the lyophilized conditioned medium in a suspension mixture, the suspension mixture comprising one or more of: saline, hyaluronic acid, silicone gel, petroleum jelly, PRP, derivative of PRP, platelet lysate, and a pharmaceutically acceptable excipient.

9. A conditioned medium prepared using the method of claim 1.

10. A conditioned medium comprising one or more bioactive factors secreted by a hair follicle-derived cell obtained non-invasively from a subject.

11. The conditioned medium of claim 9, wherein the one or more bioactive factors comprise growth factor, cytokine, chemokine, small peptide, nucleic acid, extracellular matrix molecule, extracellular vesicle, factors capable of mediating a physiological process, or any combination thereof.

12. The conditioned medium of claim 11, wherein the physiological process comprises wound-healing, cell proliferation, angiogenesis, anti-inflammation, or any combination thereof.

13. The conditioned medium of claim 9, wherein the one or more bioactive factors comprise hyaluronic acid, elastin, HAPLN1, collagens (e.g., COL1A1, COL2A1, COL3A1), keratins (e.g., KRT5, KRT19), fibronectin, EMILIN1, KGF, PDGF, bFGF, TGF-J31, HGF, EGF, VEGF, CCL19, sAXL, SDF-1, VCAM-1, MCP-1, IGF-1,
GDNF, BDNF, NRG2, PDGF, Interleukins (e.g., IL-1, IL-2, IL-6), or any combination thereof, at a concentration of about 0 pg/mL to about 1000 pg/mL.

14. The conditioned medium of claim 9, wherein the conditioned medium is substantially free of whole cells, cellular debris, and/or xenogeneic-free.

15. A composition comprising:
the conditioned medium of claim 9; and
one or more of: saline, hyaluronic acid, PRP, derivative of PRP, platelet lysate, a pharmaceutically acceptable excipient, carrier, and diluent.

16. The composition of claim 15, wherein the PRP, derivative of PRP, or platelet lysate is autologous to the hair follicle-derived cell that conditioned the conditioned medium.

17. The composition of claim 15, formulated into dosage forms selected from injection, oily suspension, hydrogel, nanogel, ointment, cream, serum, emulsion, spray, patch, gel, drop, or any combination thereof.

18. Use of the conditioned medium of claim 9 for therapeutic or cosmetic treatment.

19. The use of claim 18, wherein the therapeutic or cosmetic treatment comprises stimulating soft or connective tissue repair in a targeted tissue site, hair regeneration, skin rejuvenation, reduction or prevention of signs of skin aging, or treatment of joint-related conditions.

20. The use of claim 18, wherein the targeted tissue site is autologous to the hair follicle-derived cell that conditioned the conditioned medium.
